(19) United States
(12) Patent Application Publication
(10) Pub. No.: US 2021/0186747 A1
(43) Pub. Date: Jun. 24, 2021

(54) BREATHING ASSISTANCE ORAL APPARATUS

(71) Applicant: Oventus Medical Limited, Indooroopilly, Queensland (AU)

(72) Inventors: Christopher Patrick HART, Indooroopilly, Queensland (AU); Neil ANDERSON, Indooroopilly, Queensland (AU); Michael Leigh SLATER, Indooroopilly, Queensland (AU); Benjamin LOW, Chapel Hill, Queensland (AU); David OGLE, Indooroopilly, Queensland (AU); Vu Thua NGUYEN, Indooroopilly, Queensland (AU)

(21) Appl. No.: 17/051,898

(22) PCT Filed: May 2, 2019
(86) PCT No.: PCT/AU2019/050402

§ 371 Date: Oct. 30, 2020

(30) Foreign Application Priority Data
May 2, 2018 (AU) 2018901485

(51) Int. Cl. A61F 5/56; B29C 64/386; B33Y 80/00

(52) U.S. Cl. CPC A61F 5/566; B33Y 80/00; B29C 64/386

(57) ABSTRACT

A breathing assistance apparatus including a body shaped to be at least partially positioned within the oral cavity of a user, the body including: a bite configured to receive teeth of the user in use; an extra-oral opening that extends between lips of the user; an intra-oral opening provided in the oral cavity to allow airflow into and/or out of a posterior region of the oral cavity; and, at least one airway wall configured to define in combination with the bite an enclosed airway extending between the extra-oral and intra-oral openings.

### BACKGROUND OF THE INVENTION

The present invention relates to a breathing assistance oral apparatus, and in particular an oral apparatus for providing breathing assistance during sleeping, as well as a method of manufacturing a breathing assistance oral apparatus.

### Description of the Prior Art

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Poor quality or ineffective breathing is an issue which can affect the performance of people in their day to day activities either while they are awake and/or when they are asleep. While awake this can be less optimal performance in activities such as sport or even while performing everyday tasks. While asleep breathing disorders can lead to snoring and/or sleep apnoea.

Snoring arises due to vibration of soft tissues within the respiratory pathways of an individual, and is typically caused by obstructed air movement during breathing while sleeping. Snoring can arise from a range of different physical causes such as blocked sinuses, and typically occurs when the muscles of the upper throat relax during sleep.

Snoring can also be associated with Obstructive Sleep Apnoea (OSA), which is caused by obstruction of the upper airway and results in repetitive pauses in breathing during normal sleep. Individuals having OSA often suffer from daytime sleepiness and fatigue associated with significant levels of sleep disturbance, whilst a partners sleep patterns are also often disturbed by associated snoring.

Current therapy for treatment of OSA can include lifestyle changes, the use of mechanical devices, such as oral or nasal devices that augment the airway, surgical procedures to enlarge and stabilize the airway during sleep, and continuous or variable positive airway pressure (PAP) devices.

However, surgical procedures can be severe and are not therefore widely used unless absolutely necessary. Whilst PAP devices have had a positive impact, these can be uncomfortable to wear for prolonged time periods, are expensive, and are often noisy, which can in turn lead to additional sleep disturbance. As a result, surgery and PAP treatments have limited application in treating sleep apnoea, and are not generally considered appropriate treatment for snoring.

It has been shown that approximately 30-50% of continuous variable positive airway (CPAP) device users are non-compliant users with 2 years of starting their treatment. CPAP systems deliver airflow to a mask which the user typically wears over their mouth and nose. CPAP masks suffer from several drawbacks including leakage and discomfort and often users experience a degree of claustrophobia whilst wearing the mask.

Furthermore, as CPAP systems must supply air at sufficient pressure to maintain an airway and act as a pneumatic splint, relatively high pressures are typically required. In addition, high flow rates are required as the mask supplies all of the air for a user during inhalation. In order to achieve such high pressures and flow, relatively large and noisy pumps such as air blowers are conventionally used.

In terms of other mechanical devices, nasal devices have been used that dilate the nasal airway using traction or splinting. However, these have typically not had much success and can be uncomfortable for a user.

US2004/194787 describes an anti-snoring device that includes a flexible hollow tube for insertion into the user's mouth, having proximal and distal ends and an outer perimeter. The tube includes an extra oral segment at its proximal end, an intraoral segment at its distal end and an intermediate segment extending therebetween. The extra oral and intraoral segments each include at least one opening. The extra oral segment is for extending beyond the user's outer lips, the intermediate segment is of a sufficient length for extending along the buccopharyngeal pathway of the user's mouth, and the intraoral segment is of a sufficient length for extending beyond a retromolar space in the user's mouth, into the oropharynx and terminating between the posterior tongue and the soft palate. The anti-snoring device also includes a stop extending from the outer perimeter of the tube on the intraoral segment for securing the intraoral segment within the user's oropharynx. However, whilst this arrangement can assist in providing an additional airway, and hence reduce snoring and apnoea events, it can be uncomfortable to wear and can move within the mouth during use, which can reduce device effectiveness and in turn lead to additional breathing problems.

US2005/150504 describes a device which is removably insertable in the mouth for facilitating breathing while sleeping which provides a clear unobstructed airway by protrusive positioning of the mandible and/or delivery of pressurized air to the back of the mouth. The device has upper and lower tooth-contacting members and an airway defined between them, and is designed specifically for use with CPAP machines. Consequently, this device can only be used in limited circumstances, where CPAP machines are available, and is only used in the treatment of sleep apnoea.

WO2012/155214 describes an apparatus for providing breathing assistance, the apparatus including a body including a recess for receiving teeth of a user to thereby position the body within an oral cavity of the user, a first opening extending beyond lips of a user to allow air from outside the oral cavity to be drawn in through the opening, a second opening provided in the oral cavity to allow air to be directed into a posterior region of the oral cavity and a channel connecting the first and second openings, the channel extending through at least part of a buccal sulcus of the user.

WO2017/020079 provides an apparatus for providing breathing assistance, the apparatus including a body for positioning within an oral cavity of a user, the body defining, at least one first opening for allowing airflow between lips of the user, two second openings provided in the oral cavity to allow air flow into and out of a posterior region of the oral cavity, two channels, each channel connecting a respective second opening to the at least one first opening and each channel passing at least one of at least partially along the buccal cavity and at least partially between the teeth to thereby provide an airway for the user, the airway at least partially bypassing the nasal passage to thereby act to replicate a healthy nasal passage and pharyngeal space and a tongue retaining portion including a cavity for receiving a portion of a tongue of the user, in use, wherein the tongue retaining portion is configured to retain the tongue in an extended position to project at least partially between the tooth of the user.

Reference to any prior art in the specification is not, and should not be taken as, an acknowledgment or any form of suggestion that this prior art forms part of the common general knowledge in any jurisdiction or that this prior art could reasonably be expected to be understood, regarded as relevant and/or combined with other pieces of prior art by a person skilled in the art.

### SUMMARY

In one broad form an aspect of the present invention seeks to provide a breathing assistance apparatus including a body shaped to be at least partially positioned within the oral cavity of a user, the body including: a bite configured to receive teeth of the user in use; an extra-oral opening that extends between lips of the user; an intra-oral opening provided in the oral cavity to allow airflow into and/or out of a posterior region of the oral cavity; and, at least one airway wall configured to define in combination with the bite an enclosed airway extending between the extra-oral and intra-oral openings.

In one embodiment the airway is configured to be positioned at least one of: substantially between the user's teeth in use; and, only between the user's teeth in use.

In one embodiment the at least one airway wall includes inner and outer airway side walls extending from an airway base wall to the bite.

In one embodiment the at least one airway wall is at least partially deformable to accommodate relative movement of a user's mandibular and maxillary teeth.

In one embodiment the bite includes inner and outer bite side walls extending from a bite base.

In one embodiment the bite is shaped to conform to an arch of the user's teeth.

In one embodiment the at least one airway wall defines an open channel in engagement with the bite to form the enclosed airway.

In one embodiment the at least one airway wall is coupled to the bite using at least one of: welding; adhesive; chemical bonding; and, mechanical bonding.

In one embodiment the at least one airway wall and the bite are formed integrally using additive manufacturing.

In one embodiment the apparatus includes at least one of: a maxillary bite configured to receive maxillary teeth of the user in use; and, a mandibular bite configured to receive mandibular teeth of the user in use.

In one embodiment the apparatus includes at least one of: a first body including the maxillary bite; and, a second body including the mandibular bite.

In one embodiment the apparatus includes a coupling that allows a relative position of the first and second bodies to be adjusted.

In one embodiment the coupling includes an arm extending between first and second arm mountings provided on the first and second bodies respectively, and wherein a relative position of the first and second bodies is adjusted based on a length of the arm.

In one embodiment the apparatus includes: a first body including: a maxillary bite; the extra-oral opening; the intra-oral opening; and, the at least one airway; and, a second body including the mandibular bite.

In one embodiment the extra-oral opening is defined by a tubular body protruding forwardly from the body.

In one embodiment the body includes at least two channels, each channel connecting a respective intra-oral opening to the extra-oral opening.

In one embodiment the body is made of at least one of: nylon; a thermosetting polymer; a thermoplastic polymer; silicone; an elastomer; polycarbonate; acrylonitrile butadiene styrene; polyvinylsiloxane; polyurethane; and, ethylvinylacetate.

In one embodiment the at least one airway wall and the bite are formed from different materials.

In one broad form an aspect of the present invention seeks to provide a method of manufacturing a breathing assistance apparatus for a user, the method including: obtaining shape information indicative of a shape of at least one of the user's dental arches; manufacturing a body shaped to be at least partially positioned within the oral cavity of a user, the body including: a bite configured to receive teeth of the user in use; an extra-oral opening that extends between lips of the user; an intra-oral opening provided in the oral cavity to allow airflow into and/or out of a posterior region of the oral cavity; and, at least one airway wall configured to define in combination with the bite an enclosed airway extending between the extra-oral and intra-oral openings.

In one embodiment the method includes: manufacturing the bite; manufacturing the at least one airway wall to define an open channel: and, coupling the at least one airway wall to the bite to form the enclosed airway.

In one embodiment the method includes coupling the at least one airway wall to the bite using at least one of: welding; adhesive; chemical bonding; and, mechanical bonding.

In one embodiment the method includes integrally forming the at least one airway wall and the bite using additive manufacturing.

In one embodiment the method includes manufacturing the bite using the shape information.

In one embodiment the method includes manufacturing the bite using additive manufacturing.

In one embodiment the method includes manufacturing the body using at least one of: nylon; a thermosetting polymer; a thermoplastic polymer; silicone; an elastomer; polycarbonate; acrylonitrile butadiene styrene; polyvinylsiloxane; polyurethane; and, ethylvinylacetate.

In one embodiment the method includes manufacturing the at least one airway wall and the bite are formed from different materials.

In one embodiment the method includes manufacturing at least one of a first body including a maxillary bite shaped to conform to maxillary teeth of the user; and, a second body including a mandibular bite shaped to conform to mandibular teeth of the user.

In one embodiment the method includes deriving the shape information from at least one of: a dental impression; a series of photos of the user's teeth; a scan of at least part of the user's oral cavity; a CT scan of at least part of the user's oral cavity; a 3D scan of the user's teeth; and, cone beam imaging.

In one embodiment the series of photos of the patient's mouth or impression taken with a smart phone and the photos are then loaded into a software program to derive a 3D image including an STL file.

In one embodiment the shape information includes dimensions of at least one of: at least part of the oral cavity of the user; and, at least part of dental arches of the user.

In one embodiment the method includes: obtaining template data representing a bite design; modifying the bite design using the shape information; generating modified template data using the modified bite design; and, manufacturing the body using the modified template data.

In one embodiment the modified template data is in the form of a print file for use in an additive manufacturing machine.

It will be appreciated that the broad forms of the invention and their respective features can be used in conjunction, interchangeably and/or independently, and reference to separate broad forms is not intended to be limiting.

### BRIEF DESCRIPTION OF THE DRAWINGS

Various examples and embodiments of the present invention will now be described with reference to the accompanying drawings, in which:

FIG. 1A is a schematic perspective topside view of an example of a breathing assistance apparatus;

FIG. 1B is a schematic rear topside view of the breathing apparatus of FIG. 1A;

FIG. 1C is a schematic front view of the breathing apparatus of FIG. 1A;

FIG. 1D is a schematic rear view of the breathing apparatus of FIG. 1A;

FIG. 1E is a schematic plan cutaway view of the breathing apparatus of FIG. 1A taken along the line A-A′ of FIG. 1D;

FIG. 1F is a schematic plan view of the breathing apparatus of FIG. 1A;

FIG. 1G is a schematic underside view of the breathing apparatus of FIG. 1A;

FIG. 1H is a schematic rear cutaway view of the breathing apparatus of FIG. 1A taken along the line B-B′ of FIG. 1F; and,

FIG. 2 is a schematic front view of an alternative breathing apparatus.

### DETAILED DESCRIPTION OF THE PREFERRED EMBODIMENTS

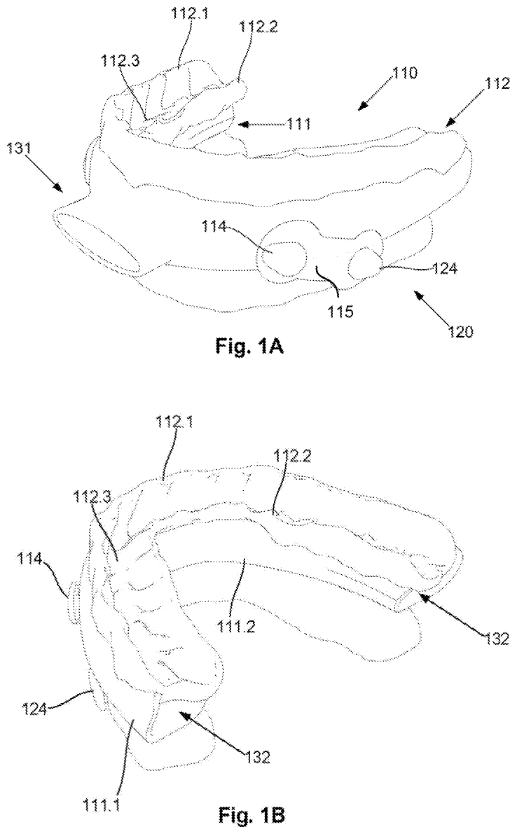

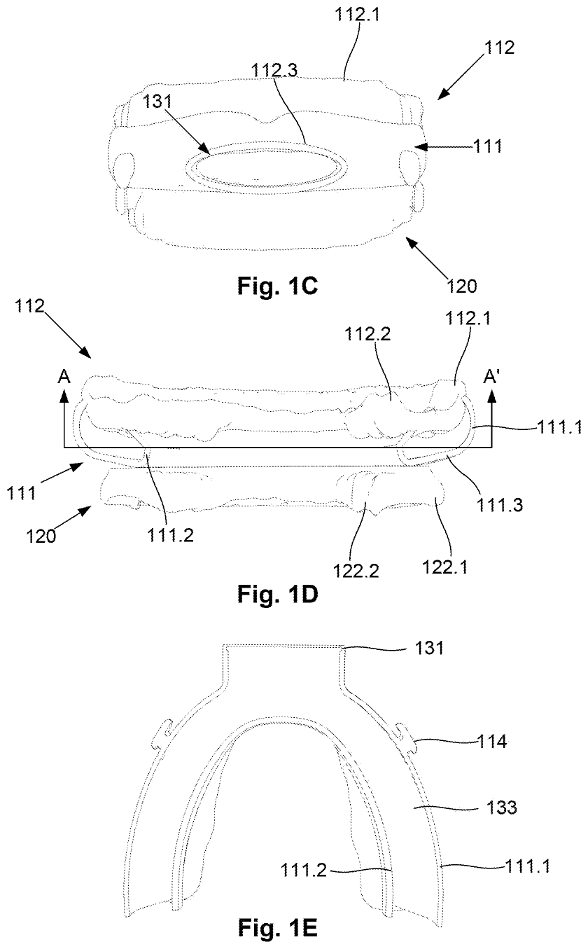

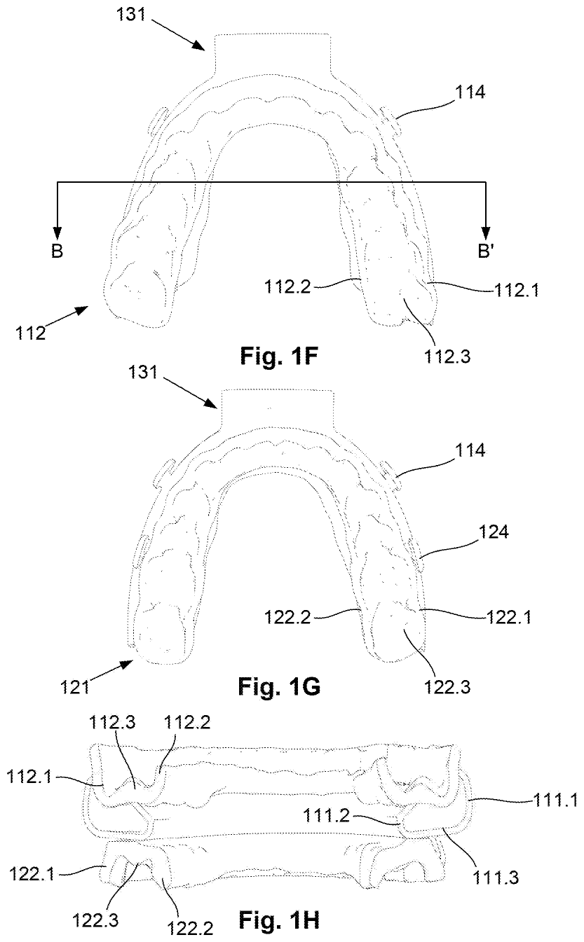

An example of the breathing apparatus will now be described with reference to FIGS. 1A to 1H.

In this example, the breathing assistance apparatus includes an oral appliance having a body that is shaped to be at least partially positioned within the oral cavity of a user. The body includes a bite configured to receive teeth of the user in use. Although a single bite could be provided, more typically upper and lower bites 112, 122 are provided to receive the maxillary and mandibular teeth, respectively.

In this example, the body includes first and second upper and lower bodies 110, 120, each including a respective one of the upper and lower bites 112, 122. The bodies can be coupled together to form a single body, whilst allowing a relative position of the upper and lower bodies to be adjusted, to provide for mandibular advancement, as will be described in more detail below. However, it will be appreciated from the following description that this is not essential and a single unitary body could be provided.

The body also includes an extra-oral opening 131 that in use extends between lips of the user, and an appliance airway 133 passing through the body 110, to one or more intraoral openings 132 provided in the oral cavity, to allow airflow into and out, of a posterior region of the oral cavity. This allows air to pass through the appliance airway so that it is directed into a posterior region of the mouth through the second openings 132, thereby at least partially bypassing the nasal passage and acting to replicate a healthy nasal passage and pharyngeal space. Providing air flow directly into a posterior portion of the user's oral cavity has a number of benefits. In particular, this avoids obstructions created by the nasal cavity, soft palate and tongue which can lead to snoring and apnoea events, and helps reduce the drying effects of air flow, which can in turn lead to user discomfort. Thus, for example, nasal obstructions can be bypassed by air flow through the apparatus, thereby bypassing the nasal airway or adding to it in the case of a partial obstruction. Furthermore, air flowing below or on both sides of the soft palette helps prevent collapse of the soft palate, which can in turn lead to additional obstruction.

The airway 133 is constructed from at least one airway wall 111.1, 111.2, 111.3, configured as an open channel which is provided in engagement with a bite 112, 122, to form an enclosed airway 133. in this example, the airway wall 111.1, 111.2, 111.3 is in engagement with the upper bite 112, but this is not essential and arrangements in which the airway is provided in engagement with a lower bite 122 could be used.

Accordingly, in the above described arrangement, the airway is defined by a combination of an open channel formed from an airway wall and a bite configured to receive the teeth of the user, so that the airway is effectively integrally formed with, and as part of, the bite. Integrally forming the airway channel using part of the bite avoids the need for a separately enclosed channel, which in turn minimises the volume of material required to manufacture the airway and bite. This in turn allows the size of the body to be minimised, which helps maintain comfort, whilst maximising the cross sectional area of the airway 133 to ensure suitable airflow can be achieved through the user. This arrangement enables the body to be manufactured using polymer materials, for example using an additive manufacturing process such as laser sintering of nylon, which would not otherwise achievable without resulting in a device that is unnecessary bulky, and hence uncomfortable.

A further advantage of this arrangement is that this minimises the overall height of the internal airway by avoiding the need for an upper enclosing wall, in turn allowing the airway to be accommodated almost solely between the maxillary and mandibular teeth. This avoids the need for an airway which passes along the buccal sulcus between the cheeks and the gums which can in turn result in further discomfort for a user.

A number of further features will now be described.

In one example, the airway wall includes inner and outer side walls 111.1, 111.2 which extend from a base wall 111.3 towards the bite 112. The side walls and base wall 111.1. 111.2, 111.3 can be made from discrete separate elements, bonded using welding adhesive or the like. However, more typically the airway wall is a substantially continuous member shaped to define the inner and outer side walls and base wall, meaning delineation of these features is largely for the purpose of illustration.

In one example, the at least one airway wall, and more particularly the inner and outer airway side walls 111.1, 111.2 are deformable to thereby accommodate relative movement of the user's mandibular and maxillary teeth. In particular, if the user moves their teeth together, for example by clenching their jaw, the airway side walls 111.1, 111.2 can bend outwardly, compressing the height of the airway, whilst maintaining a substantially constant airway cross-sectional area. This ensures airflow is maintained while absorbing the clenching force of the user's teeth. Similarly, relative lateral movement of the mandibular and maxillary teeth can be accommodated through flexure of the side walls. This is particularly useful for users suffering from bruxism, and in particular, reduces stress on the user's teeth whilst accommodating lateral and clenching motions without damaging the oral appliance.

This can be achieved by manufacturing the airway from a suitable material, such as a polymer, for example by manufacturing using laser sintering of a nylon material, or injection moulding of a polymer, such as a thermosetting polymer, a thermoplastic polymer, silicone, an elastomer, polyvinylsiloxane, polyurethane, ethylvinylacetate, polycarbonate, acrylonitrile butadiene styrene, or a combination of these materials. However, this is not essential, and alternatively the body can be made of metal, such as titanium, or other materials, depending on the preferred implementation.

In one example, the bite includes inner and outer bite side walls extending from a bite base. In this particular example, the body includes a first maxillary bite 112 including outer and inner side walls 112.1, 112.2 extending upwardly from a bite base 112.3, as well as a second mandibular bite including outer and inner side walls 122.1, 122.2 extending downwardly from a bite base 122.3. Thus, the bites effectively define open channels into which a user's teeth can be positioned, to thereby retain the oral appliance in position within the oral cavity. As described below, these are typically customised for a specific user, to thereby ensure a suitable fit, and to maintain comfort in use.

As previously mentioned, whilst the body could be a single unitary body, more typically the body can be manufactured as separate upper and lower bodies 110, 120, which engage separately with the upper and lower jaw. This allows different sizes of body to be used, as well as allowing a relative position of the upper and lower bodies to be adjusted and thereby adjusting a degree of mandibular advancement.

In this regard, mandibular advancement can assist in holding open the user's airway, which in turn can reduce snoring. For example, temporomandibular joint disorder (TMD) arises when the upper and lower jaws are misaligned. This may be naturally occurring or can result from injury, or the like. Regardless, such jaw misalignment tends to contribute to airway obstructions by changing the shape of the upper airway, and moving the tongue towards the posterior of the oral cavity, which can in turn exacerbate issues associated with OSA and snoring. Accordingly, by allowing the relative position of the first and second bodies to be adjusted, this allows the jaws of the user to be aligned thereby reducing the effects of TMD, and hence further reducing the likelihood of snoring and OSA.

In this example, the bodies 110, 120 include a coupling that allows relative position of the first and second bodies to be adjusted. Whilst any suitable arrangement could be used, in this example this is achieved using first and second mountings 114, 124 that project outwardly from sides of the first and second bodies 110, 120 with these being interconnected via respective arms 115 (depicted in FIG. 1A, and omitted from FIGS. 1B to 1H). A relative position of the first and second bodies can then be adjusted based on a length of the aims, with the aims being extendible, or interchangeable, allowing different lengths of arms to be provided to achieve a desired degree of mandibular advancement as required.

In a preferred example, the first body 110 includes the maxillary bite 112, the extra and intraoral openings 131, 132 and the airway 133 while the second body only includes the mandibular bite 122. However this is not essential and it will be appreciated that other configurations could be used.

In the above described arrangement, the extra-oral opening is defined by a tubular body protruding forwardly from the body. This allows the tubular body to extend beyond the lips of the user, as well as allowing this to be used to couple to a connector, for connection of a positive airway pressure (PAP) machine, or to accommodate valves, humidity exchangers, filters or the like.

The body typically includes at least two channels, each channel connecting a respective intra-oral opening to the extra-oral opening, although this is not essential and other arrangements can be used.

To ensure the device is comfortable to use, at least the bites are typically shaped to conform to at least an arch of the user's teeth. In this example, a method of manufacturing a breathing assistance apparatus involves obtaining shape information indicative of a shape of at least the user's dental arches and then manufacturing the body including, the bite, the extra-oral opening, the intra-oral opening and airway wall.

In particular, in a preferred embodiment the bite(s) are customised to a user for example forming the bite(s) through an additive manufacturing process utilising shape information indicative of a shape of at least the user's dental arches. Having made the bite, this is then coupled to the airway wall, for example using welding, adhesive, chemical bonding, or mechanical bonding, or the like, to thereby form the enclosed airway.

As part of this process, the airway wall can also be customised, or alternatively can be manufactured using one of a number of standard template sizes, selected to provide the best fit with the bite. However, this is not essential, and it will be appreciated that the airway and bite could be integrally formed, for example using 3D printing or another similar approach.

The shape information typically includes dimensions of at least part of the oral cavity of the user or the dental arches of the user, and can be derived from any one or more of a dental impression, a series of photos of the user's teeth, a scanned part of the user's oral cavity, a CT scan of parts of the user's oral cavity, a 3D scan of the user's teeth and/or cone beam imaging. In one particular example the shape information is derived from a series of photos of the patient's mouth or an impression which is taken by a smart phone with the photos being loaded into a software program in order to derive a 3D image including an STL file which can then be used to form the bites through additive manufacturing. In one example this is achieved by obtaining template data representing a bite design, modifying the bite design using shape information and then generating modified template data using the modified bite design with this being used to manufacture the bites.

The body can be made from any suitable material, including high strength polymers, plastics, VeroGlaze (MED620) dental material, or the like. This can be achieved using additive printing, injection moulding or any other suitable technique. For example, the body 110 can be manufactured using laser sintering of a nylon material, or injection moulding of a polymer, such as a thermosetting polymer, a thermoplastic polymer, silicone, an elastomer, polyvinylsiloxane, polyurethane, ethylvinylacetate, polycarbonate, acrylonitrile butadiene styrene, or a combination of these materials. It will be appreciated that, in some examples, the bite(s) and the airway wall may be manufactured from different materials. For example, the airway wall may be manufactured from a harder material, such as a nylon or polycarbonate material, to ensure a sufficient cross sectional area of the airway is maintained. In contrast, the bite(s) may be manufactured from a softer material, e.g. an elastomer such as urethane or ethylvinylacetate, for greater comfort when contacting the user's teeth.

The body 110 can be coated with a medical grade polymer and in one example, a medical grade elastomer, such as silicone or polyurethane, epoxy or parylene, for improved comfort as well as ensuring biocompatability. In one example, the coating can include an Active Composite Guidance, which is a 3 dimensional composite resin with different shapes and sizes and which can be bonded to the body to ensure accurate positioning of the body with respect to the user's teeth. Coatings can be applied to the body using any suitable technique, such as dip coating, vapour coating, or spray coating the body, thereby ensuring all exposed surfaces, including internal surfaces of the channels, are coated. As part of this process, this can include applying primers to the body prior to coating, thereby ensuring the coating adheres to the body. As an alternative, or in addition to coating, at least part of the body can be polished using at least one of mechanical and electrochemical polishing.

In one example, the bites are customised as described above, before a coating layer of mouldable polymer, such as silicone, is applied, allowing for final finishing of the bite shape. In particular, this can be achieved by having the user wear the appliance as the material moulds to the shape of the user's teeth before it is solidified. For example, this could include UV curing, using a thermosetting material, a thermosetting polymer, a thermoplastic polymer, silicone, an elastomer, polyvinylsiloxane, polyurethane, ethylvinylacetate, polycarbonate, acrylonitrile butadiene styrene, or a combination of these materials, could be used.

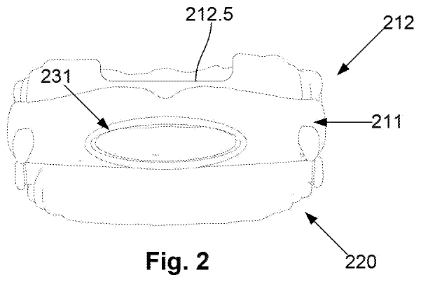

A further example arrangement is shown in FIG. 2.

In this example, the appliance is broadly similar to that described above and includes first and second upper and lower bodies 210, 220, each including a respective one of the upper and lower bites 212, 222. However, in this example, the upper bite 212 includes a cut-out section 212.5, which reduces the volume of material in the upper bite. In this example, the cut-out section is provided in a region that would be positioned between the user's incisors and lips, in use, which can therefore reduce the space taken up in this region of the user's mouth, which in turn provides additional comfort. It will also be appreciated that cut-outs may be provided in other areas as needed, for example to reduce the overall volume of material used, and/or to increase user comfort.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers. As used herein and unless otherwise stated, the term “approximately” means±20%.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

It will of course be realised that whilst the above has been given by way of an illustrative example of this invention, all such and other modifications and variations hereto, as would be apparent to persons skilled in the art, are deemed to fall within the broad scope and ambit of this invention as is herein set forth.

1. A breathing assistance apparatus including a body shaped to be at least partially positioned within the oral cavity of a user, the body including:
a) a bite configured to receive teeth of the user in use;
b) an extra-oral opening that extends between lips of the user;
c) an intra-oral opening provided in the oral cavity to allow airflow into and/or out of a posterior region of the oral cavity; and,
d) at least one airway wall configured to define in combination with the bite an enclosed airway extending between the extra-oral and intra-oral openings.

2. An apparatus according to claim 1, wherein the airway is configured to be positioned at least one of:
a) substantially between the user's teeth in use; and,
b) only between the user's teeth in use.

3. An apparatus according to claim 1, wherein the at least one airway wall includes inner and outer airway side walls extending from an airway base wall to the bite.

4. An apparatus according to claim 1, wherein the at least one airway wall is at least partially deformable to accommodate relative movement of a user's mandibular and maxillary teeth.

5. An apparatus according to claim 1, wherein the bite includes inner and outer bite side walls extending from a bite base.

6. An apparatus according to claim 1, wherein the bite is shaped to conform to an arch of the user's teeth.

7. An apparatus according to claim 1, wherein the at least one airway wall defines an open channel in engagement with the bite to form the enclosed airway.

8. An apparatus according to claim 7, wherein the at least one airway wall is coupled to the bite using at least one of:
a) welding;
b) adhesive;
c) chemical bonding; and,
d) mechanical bonding.

9. An apparatus according to claim 1, wherein the at least one airway wall and the bite are formed integrally using additive manufacturing.

10. An apparatus according to claim 1, wherein the apparatus includes at least one of:
a) a maxillary bite configured to receive maxillary teeth of the user in use; and,
b) a mandibular bite configured to receive mandibular teeth of the user in use.

11. An apparatus according to claim 10, wherein the apparatus includes at least one of:
a) a first body including the maxillary bite; and,
b) a second body including the mandibular bite.

12. An apparatus according to claim 11, wherein the apparatus includes a coupling that allows a relative position of the first and second bodies to be adjusted.

13. An apparatus according to claim 12, wherein the coupling includes an arm extending between first and second arm mountings provided on the first and second bodies respectively, and wherein a relative position of the first and second bodies is adjusted based on a length of the arm.

14. An apparatus according to claim 1, wherein the apparatus includes:
a) a first body including:
i) a maxillary bite;
ii) the extra-oral opening;
iii) the intra-oral opening; and,
iv) the at least one airway; and,
b) a second body including the mandibular bite.

15. An apparatus according to claim 1, wherein the extra-oral opening is defined by a tubular body protruding forwardly from the body.

16. An apparatus according to claim 1, wherein the body includes at least two channels, each channel connecting a respective intra-oral opening to the extra-oral opening.

17. An apparatus according to claim 1, wherein the body is made of at least one of:
a) nylon;
b) a thermosetting polymer;
c) a thermoplastic polymer;
d) silicone;
e) an elastomer;
f) polycarbonate;
g) acrylonitrile butadiene styrene;
h) polyvinylsiloxane;
i) polyurethane; and,
j) ethylvinylacetate.

18. An apparatus according to claim 1, wherein the at least one airway wall and the bite are formed from different materials.

19. A method of manufacturing a breathing assistance apparatus for a user, the method including:
a) obtaining shape information indicative of a shape of at least one of the user's dental arches;
b) manufacturing a body shaped to be at least partially positioned within the oral cavity of a user, the body including:
i) a bite configured to receive teeth of the user in use;
ii) an extra-oral opening that extends between lips of the user;
iii) an intra-oral opening provided in the oral cavity to allow airflow into and/or out of a posterior region of the oral cavity; and,
iv) at least one airway wall configured to define in combination with the bite an enclosed airway extending between the extra-oral and intra-oral openings.

20. A method according to claim 19, wherein the method includes:
a) manufacturing the bite;
b) manufacturing the at least one airway wall to define an open channel; and,
c) coupling the at least one airway wall to the bite to form the enclosed airway.

21. A method according to claim 19, wherein the method includes coupling the at least one airway wall to the bite using at least one of:
a) welding;
b) adhesive;
c) chemical bonding; and,
d) mechanical bonding.

22. A method according to claim 19, wherein the method includes integrally forming the at least one airway wall and the bite using additive manufacturing.

23. A method according to claim 19, wherein the method includes manufacturing the bite using the shape information.

24. A method according to claim 19, wherein the method includes manufacturing the bite using additive manufacturing.

25. A method according to claim 19, wherein the method includes manufacturing the body using at least one of:
a) nylon;
b) a thermosetting polymer;
c) a thermoplastic polymer;
d) silicone;
e) an elastomer;
f) polycarbonate;
g) acrylonitrile butadiene styrene;
h) polyvinylsiloxane;
i) polyurethane; and,
j) ethylvinylacetate.

26. A method according to claim 19, wherein the method includes manufacturing the at least one airway wall and the bite are formed from different materials.

27. A method according to claim 19, wherein the method includes manufacturing at least one of:
a) a first body including a maxillary bite shaped to conform to maxillary teeth of the user;
b) a second body including a mandibular bite shaped to conform to mandibular teeth of the user.

28. A method according to claim 19, wherein the method includes deriving the shape information from at least one of:
a) a dental impression;
b) a series of photos of the user's teeth;
c) a scan of at least part of the user's oral cavity;
d) a CT scan of at least part of the user's oral cavity;
e) a 3D scan of the user's teeth; and,
f) cone beam imaging.

29. A method according to claim 28, wherein the series of photos of the patients mouth or impression taken with a smart phone and the photos are then loaded into a software program to derive a 3D image including an STL file.

30. A method according to claim 19, wherein the shape information includes dimensions of at least one of:
a) at least part of the oral cavity of the user; and,
b) at least part of dental arches of the user.

31. A method according to claim 19, wherein the method includes:
a) obtaining template data representing a bite design;
b) modifying the bite design using the shape information;
c) generating modified template data using the modified bite design; and,
d) manufacturing the body using the modified template data.

32. A method according to claim 31, wherein the modified template data is in the form of a print file for use in an additive manufacturing machine.
